(19) United States
(12) Patent Application Publication
(10) Pub. No.: US 2023/0143790 A1
(43) Pub. Date: May 11, 2023

(54) METHOD FOR THE AEROBIC AND ANAEROBIC CULTIVATION OF MICROORGANISMS, METHOD FOR THE PRODUCTION OF A PREPARATION FOR CLEANING RADIOACTIVE LIQUIDS AND RADIOACTIVELY CHARGED SURFACES, METHOD FOR CLEANING RADIOACTIVE LIQUIDS AND METHOD FOR CLEANING RADIO-ACTIVELY CHARGED SURFACES

(71) Applicant: Sylvia SCHREIBER, Dornbirn (AT)

(72) Inventors: Albina Aleksandrovna KORNILOVA, Moscow (RU); Vladimir Ivanovich VYSOTSKII, Kiev (UA)

(73) Assignee: Sylvia SCHREIBER, Dornbirn (AT)

(21) Appl. No.: 17/907,840

(22) PCT Filed: Feb. 24, 2021
(86) PCT No.: PCT/EP2021/054616

§ 371 Date: Aug. 29, 2022

(30) Foreign Application Priority Data
Feb. 29, 2020 (DE) 10 2020 001 337.6

(51) Int. Cl. G21F 9/18; G21F 9/28

(52) U.S. Cl. CPC G21F 9/18; G21F 9/28

(57) ABSTRACT

The invention relates to a method for aerobic and anaerobic cultivation of microorganisms. The invention also relates to a method for producing a preparation for cleaning radioactive liquids and radioactively charged surfaces. Likewise, the invention further relates to a method for cleaning radioactive liquids and radioactively charged surfaces.

### CROSS-REFERENCE TO RELATED APPLICATIONS

This application is a national stage under 35 U.S.C. §371 of PCT Patent Application PCT/EP2021/054616 filed on 24 Feb. 2021, which is pending and which is hereby incorporated by reference in its entirety for all purposes. PCT/EP2021/054616 claims priority to German Patent Application 10 2020 001 337.6 filed on 29 Feb. 2020, which is hereby incorporated by reference in its entirety for all purposes.

### BACKGROUND OF THE INVENTION

### 1. Field of the Invention

The invention is directed to a method for the aerobic and anaerobic cultivation of microorganisms, a method for the production of a preparation for cleaning radioactive liquids and radioactively charged surfaces, and . a method for cleaning radioactive liquids and radioactively charged surfaces.

### 2. Discussion of the Related Art

Radioactive liquids may accrue in connection with various, particularly technical processes. The largest source of radioactive liquids is to be found in connection with the operation of nuclear power plants. The enormous amounts of radioactive liquids accruing as waste in nuclear power plants are a considerable problem for the operators of the nuclear power plants on the one hand and for life on Earth on the other hand. On the one hand, the radioactive liquids have to be temporarily stored in the area of the nuclear power plants. A final storage of the radioactive liquids has to take place so that storage times are reached which considerably exceed the half-life of the involved radioactive isotopes without risk. When thinking of one of the most common radioactive isotopes encountered in the operation of nuclear power plants, namely ¹³⁷Cs and its half-life of more than 30 years, it is easy to realise how high the challenges are. While radioactive solids have a small volume relative to their radioactivity and generally pose just a low risk of chemical nature, radioactive liquids have a large volume so that high storage capacities are required, and the liquids frequently tend to chemically attack the containers in which they are accommodated and to leak from them in the worst case. Then, it takes very little until the radioactive liquid is distributed in an uncontrolled manner, for example via the ground water, so that extreme damage is caused to people, animals, and the environment in general.

Already, concepts were developed how to remove the radioactive substances from the liquid to thereby produce a solid which is relatively easy to store and has precisely this radioactivity. What is known are chemical processes on the basis of an ion exchange as well as microbiological systems using radioactive substances for their growth within the scope of their development. Biological systems are insofar superior to the classical systems as they can develop a high selectivity with regard to the absorption of radioactive substances which is not necessarily guaranteed in classical chemical systems.

Further Background: On Earth, about 400 nuclear power plants are operated. In the process, a large amount of dangerous radiotoxic waste is generated. Due to the risks caused thereby, deactivation methods are considered. A deactivation of radioactive waste is generally highly complicated, cost intensive, and time consuming. Radioactively contaminated water must not end up in waste water, contaminated ground has to be removed and disposed of. Final disposal sites have to be set up so that the radioactive substances do not reach the environment and that the radiation emitted by them will not reach people and the biosphere. Prior to the final storage, the radioactive waste should be brought into a form safe for storage. Highly radioactive liquid waste is cast in steel moulds or ingot moulds with molten glass. If these containers become brittle or get damaged under the exposure to radiation and heat an environmental contamination is possible. In this case, radioactive gases may reach the atmosphere. The highest risk arises when the radioactive substances reach the ground water. The probability of such a catastrophe depends on the geological stability of the final disposal site, when it is located in a salt mine on the ground water flows surrounding the salt mine, and on the potential changes of the salt structure by radiation and heat. The containers containing radioactive substances should be permanently stored in locations where they can neither be damaged by water nor by earthquakes or other influences. Here, it should be taken into consideration that the high heat emission produced during the decay of radioactive substances requires an extremely efficient cooling of this waste over the course of many decades. Therefore, the final storage of radioactive substances is a global problem which is difficult to solve. None of the countries in which nuclear power is used has found a safe and permanent solution for the safe treatment of radioactive waste to date. Nuclear power plants only have temporary storage facilities for spent fuel. In addition, hundreds of thousands of tons of active water containing various radioactive isotopes are present in nuclear power plants. This water is produced in the operation of boiling water and pressurised water reactors as well as reactors of other types both during the long-term decay (deposition) of the fuel elements in the water after the expiration of the useful life and in the use of water as a heat transfer medium flowing through the reactor core. Only after a cool down time of three years in spent fuel pools of nuclear power plants, the overall activity of the fuel elements decreases by about one order of magnitude due to the decay of the most short-lived isotopes. 99% of the remaining activity are attributable to relatively long-lived isotopes of strontium, zirconium, ruthenium, caesium, iodine and rare earths. A certain activity is also generated by the isotopes of noble gases. Of particular importance is the deactivation of caesium radionuclides the proportion of which in the overall activity of liquid waste reaches 98%. In the practice of the deactivation of liquid waste of a low and medium activity level, cleaning methods involving organic ion exchangers are known. Thus, highly acidic cation exchangers and highly alkaline anion exchangers are used in cleaning reactor blow-down water. For cleaning water from a basin reactor, ion exchange filters charged with cation and anion exchangers are used. Further known are systems comprising organic cation exchanger resins in Na⁺ form as well as synthetic zeolithe (type A, erionite, chabasite, phillipsite). The drawback of sorption methods for deactivating solutions of medium activity with the aid of the organic ion exchanger resins is their low radiation resistance. Consequently, there is damage to the basis, the exchange capacity decreases, and the exchange kinetics deteriorate. The other disadvantage of the organic resins is their low selectivity, particularly with regard to caesium radionuclides such as ¹³⁷Cs and ¹³⁴Cs.

### SUMMARY OF THE INVENTION

The invention is based on the object to provide appropriate microorganisms in a sufficient number to produce a preparation by which radioactive liquids and radioactively charged surfaces can be reliably, efficiently, and safely cleaned, i.e. freed from their radioactivity on this basis.

This object is solved by the features of the independent claims. Advantageous embodiments are indicated in the dependent claims.

The invention consists in a method for the aerobic cultivation of microorganisms comprising the steps of:
providing an aqueous solution having a pH value of 5.5 to 9.0 in a container,
adding a substrate in a first substrate dosage to the aqueous solution,
adding further elements to the aqueous solution,
adding an inoculant including microorganisms to the aqueous solution,
wherein, by the abovementioned steps, an initial product is provided, and wherein, subsequently, the following steps are performed:
introducing air into the initial product and/or into intermediate products developing therefrom,
varying a temperature of the initial product and/or the intermediate products developing therefrom in a range from 15 to 35° C.,
wherein, after having added the substrate in the first substrate dosage, the following steps for monitoring the cultivation are performed:
taking a reference liquid sample and determining a first concentration of organic substance in the reference liquid sample,
taking another liquid sample and determining another concentration of organic substance in the further liquid sample after the expiration of the first waiting time,
if the further concentration of organic substance is smaller than 10 percent of the first concentration of organic substance, adding substrate in another substrate dosage,
repeating the abovementioned steps for monitoring the cultivation until a sufficient amount of biomass is present in the container.

The underlying aqueous solution has a pH value of 5.5 to 9.0, this value particularly taking account of the fact that the microorganisms are “aerobic microorganisms”, i.e. suitable for an aerobic cultivation. The pH value, like all parameters discussed in the following, may be varied to thereby optimise the course of the process, be it with regard to the result of the cultivation, the rate of the cultivation, or the amount of ultimately obtained microbiological material. A substrate serving the sustenance of the microorganisms is added to the aqueous solution. Likewise, other elements are added which are required for the growth of the microorganisms or stimulate the growth of the organisms. Which elements this could be will be specified in more detail below. Important for the initial product of the cultivation is the addition of an inoculant including microorganisms. These may originate from various sources, for example from earlier cultivations obtained in an identical or similar way, or also primarily from other sources, for example from sewage treatment plants. As soon as the initial product is available it may serve the cultivation of microorganisms. Since the cultivation takes place aerobically air is introduced. Furthermore, the temperature of the initial product or the intermediate products developing therefrom is varied, a temperature range of 15 to 25° C. being instrumental for this purpose. Often, temperatures in the range of 23 to 25 degrees are optimal. The initial product or the intermediate products developing therefrom are then monitored to finally obtain a sufficient amount of biomass. To this end, samples are taken, and, depending on the obtained concentrations of organic substance at the various points in time, further substrate for sustaining the cultures is added. When finally a sufficient amount of biomass is present, it can be withdrawn for further processing.

The invention further relates to a method for the anaerobic cultivation of microorganisms comprising the steps of:
providing an aqueous solution having a pH value of 4.5 to 7.5 in a container,
adding a substrate in a first substrate dosage to the aqueous solution,
adding further elements to the aqueous solution,
adding an inoculant including microorganisms to the aqueous solution,
wherein, by the abovementioned steps, an initial product is provided, and wherein, subsequently, the following steps are performed:
hermetically sealing the container,
varying a temperature of the initial product and/or the intermediate products developing therefrom in a range from 40 to 80° C.,
wherein, after having added the substrate in the first substrate dosage, the following steps for monitoring the cultivation are performed:
taking a reference liquid sample and determining a first concentration of organic substance in the reference liquid sample,
taking another liquid sample and determining another concentration of organic substance in the further liquid sample after the expiration of the first waiting time,
if the further concentration of organic substance is smaller than 10 percent of the first concentration of organic substance, adding substrate in another substrate dosage,
repeating the abovementioned steps for monitoring the cultivation until a sufficient amount of biomass is present in the container.

Compared to the method in which aerobic microorganisms are cultivated, other pH values can be used here. The value range is 4.5 to 7.5. In the method for the anaerobic cultivation, no air is introduced either, but the container in which the initial product is contained is hermetically sealed. The temperature for the cultivation of the anaerobic microorganisms is selected so as to be higher than in the cultivation of the aerobic microorganisms. It may be in a range from 40 to 80° C., preferably in a range of about 55° C. The other process steps are similar or comparable, which also applies to numerous of the preferred embodiments of the method for the aerobic or anaerobic cultivation of microorganisms specified below.

The methods are advantageously further developed particularly in that a sufficient amount of biomass is present in the containers when 50 g of wet biomass are extractable from one litre of product volume, wet biomass having a moisture content of 93 to 99 percent. Such a sufficient amount of biomass should generally be reached in the course of two weeks in the aerobic method while the aerobic method provides for a sufficient amount of biomass after about 50 days. Whether the amount is sufficient is determined by extracting biomass concentrate from the container, for example 400 ml, and centrifuging this concentrate at, for example, 4000 revolutions per minute and, for example, 3 to 8° C. The excess obtained in the centrifugation is returned. What remains is wet biomass having a moisture content from 93 to 99 percent, in case of success in an amount of more than 50 grams per litre of product volume.

Usefully, it is contemplated that the aqueous solution is based on deionised water. Since the mineral composition existing in the initial product may influence the cultivation of the microorganisms it is useful to use deionised water to clearly define the conditions prevailing in the initial product in this way, particularly on the basis of the addition of further elements influencing the growth process.

In particular, the method may be performed on the basis of using glucose and/or saccharose and/or ethanol and/or methanol as the substrate.

It is further contemplated that the first substrate dosage is added in a concentration of 0.5 g/l/d to 2 g/l/d and the further substrate dosages are added in a concentration of 0.5 g/l/d to 2 g/l/d, respectively in relation to the volume of the aqueous solution. So, when dealing with a volume of an initial product of one litre, it is, empirically, useful to add about one gram of substrate per day. Whether this is the correct amount will of course be verified by the sample collection within the scope of monitoring the cultivation.

For appropriately influencing the cultivation of the microorganisms, it is further contemplated that microelements and/or macroelements are added as further elements, the microelements being selected from the group of Mn, Mo, Zn, Cu, Co, Ni, Cl, Br, and the macroelements being selected from the group of C, H, O, N, S, K, Ca, P, Mg, Fe. Here, microelements are added in lower concentrations, and macroelements in higher concentrations.

It is particularly useful that the microelements are added in a concentration of 0.5 * 10⁻⁵ g/l to 2 * 10⁻⁵ g/l and the macroelements in a concentration of 0.5 g/l to 2 g/l, respectively in relation to the volume of the aqueous solution.

It is preferred that the inoculant contains bacteria and/or fungi and/or protozoa.

On this basis it is useful that the inoculant is added in a concentration of 5 to 10 percent in relation to the volume of the aqueous solution. The inoculant itself preferably contains water in the range of 90 to 95 percent, the remaining components being mainly or to a large extent microorganisms. Of course, it is also possible to add the microorganisms to the aqueous solution in a significantly more concentrated form or in a considerably more diluted form. The indicated ratios have proven useful in practice.

When performing the method for the aerobic cultivation of microorganisms, it is further contemplated that air is introduced during predetermined periods of time and that outside of these predetermined periods of time, no air is introduced. This intermittent supply of air, on the one hand, establishes the required chemical composition of the preparation while, on the other hand, in the rest phases, i.e., when no air is introduced, an unobstructed development of the cultivation can take place.

It is contemplated that, during the air supply phases, the rate of introduced air in relation to one litre of the volume of the initial product is from 1 I/min to 2 I/min. For example, a supply rate of 1.5 I/min has been proven in practice.

The substances containing aerobic and/or anaerobic microorganisms produced in this way may be used to produce a preparation for cleaning radioactive liquids and radioactively charged surfaces. The aerobic and/or anaerobic microorganisms may also be obtained from other sources than via the cultivation methods described above.

Insofar, the invention consists in a method for the production of a preparation for cleaning radioactive liquids comprising the steps of:
providing a substance including aerobic microorganisms, and/or
providing a substance including anaerobic microorganisms,
wherein the substance is subjected to the following steps for washing:
centrifuging the substance for producing wet biomass,
adding an isotonic solution to the wet biomass for producing a washed substance,
wherein the steps for washing are repeated a plurality of times, if required, to thereby provide the preparation for cleaning radioactive liquids and radioactively charged surfaces.

The centrifugation of the substance is preferably performed at 4000 revolutions per minute at a temperature of 3 to 8° C. The duration of the centrifugation is usefully 8 to 12 minutes. In particular, the excess of the centrifugation may be analysed for its composition, particularly its elementary composition, and utilized for the further cultivation of biomass. The isotonic solution is preferably used in a ratio of biomass to solution of 1:4 to 1:10. By sufficiently shaking or otherwise moving the mixture for preferably 5 to 10 minutes, a washed substance without clots is obtained. The solution or mixture resulting therefrom may, in turn, be centrifuged at, for example, 4000 revolutions per minute at 3 to 8° C. for 8 to 12 minutes. Now, the excess may generally be discarded since it is not to be expected that substantial amounts of biomass remain for recultivation. The centrifuging and the supply of the isotonic solution may be repeated a plurality of times, particularly three times in practice.

According to a specific embodiment, it is contemplated that the substance including aerobic microorganisms and the substance including anaerobic microorganisms are mixed prior to being washed. Potentially, aerobic microorganisms and anaerobic microorganisms can coexist. If such a mixture of microorganisms is desired mixing the aerobic and the anaerobic microorganisms prior to centrifuging and washing suggests itself. However, of course aerobic cultures of microorganisms and anaerobic cultures of microorganisms may also be processed separately.

According to a particularly preferred embodiment, it is contemplated that, after washing, an analysis of the washed biomass is carried out. Depending on the analysis, the washed biomass can be further processed.

This may, in particular, be realised so that the preparation for cleaning radioactive liquids and radioactively charged surfaces is filled into water-permeable cartridges in portions.

Furthermore, it is possible that the preparation for cleaning radioactive liquids and radioactively charged surfaces is dried by means of lyophilisation. In this case, the preparation is available as a dry powder.

The preparation produced in this way can be used for cleaning radioactive liquids and radioactively charged surfaces.

In particular, the invention consists in a method for cleaning radioactive liquids comprising the steps of:
providing radioactive liquid to be cleaned in a reactor,
adding a preparation for cleaning radioactive liquids produced as described above into the reactor,
adding a substrate into the reactor,
adding elements selected the from the group of K, Ca, P, S, N, Zn, Co, Mn, Cl, Cu, Mo, Ni, Se, B, Fe,
wherein, by the abovementioned steps, a working medium is provided, and wherein, further, the following step is performed:

removing cleaned radioactive liquid from the reactor.

The cleaning of the radioactive liquid is based on the fact that radioactive isotopes are transferred from the liquid into the microorganisms, namely by the microorganisms incorporating these radioactive isotopes in their cell structures while growing. Since the growth the microorganisms and particularly their selectivity with regard to specific radioactive isotopes can be influenced by adding the indicated elements the cleaning process can be encouraged by adding these elements. In contrast to the classical chemical processes, growing microbiological systems can ensure an extremely high selectivity and completeness of the extraction of various chemical elements (among them also radioisotopes) from various media. Microorganisms store metals by accumulation on the surface of the cell or in their interior. In the process, the storage of metals in the microorganisms does not only function in case of low metal concentrations in water solutions, but also in case of extremely high concentrations. This storage results in that the metal concentrations in the biomass of microorganisms can be higher than in the environment by a plurality of orders of magnitude. One and the same strain of microorganisms is capable of efficiently storing metals to be found in various groups of the periodic system. Such a distinct non-specificity in the storage of metals by the microorganisms indicates that physicochemical interaction mechanisms prevail between microorganisms and metals. In most cases, the storage of metals takes place by deposition on the cell surface. The metals may bond to capsular polysaccharides, a plurality of metals being stored in one capsule at the same time. The storage of metals on the cell surface may take place by the formation of a complex compound of metals and cell membrane proteins. Many metals form inseparable compounds on the cell surface, for example metal sulphides. The formation of inseparable compounds is sometimes also linked to the activity of ferments. The storage of the metals on the surface of the microorganisms is also realised by a bond to cell wall structures. The metals may not only be bound by the cell surface structures, but also stored in the cell interior. Obviously, the storage of the metals within the cells is associated with the function of transport systems. Here, both the active and the passive transport are involved in the metal extraction. After the metals have entered the cells, they may bond to cytoplasmatic proteins and interior membrane structures or form inseparable products within the cell. Although the metals are stored in the microorganisms with a clear spatial localisation in most cases, they are sometimes distributed to various locations, e.g. in the cell wall, in the membrane, and in the cytoplasm. Compared to the existing methods which make use of chemical reagents and synthetic sorbent substances the price of the biological method is many times lower due to the low costs of the production of the biological cultures and the facilities required for their use.

It is advantageous that a selection from the group of K, Ca, P, S, N, Zn, Co, Mn, Cl, Cu, Mo, Ni, Se, B, Fe of added elements is depleted of one or more elements which are to be understood to be biochemical equivalents of the elements on which the radioactivity of the radioactive liquid is based. In the selection of the elements to be added, those which can be referred to as biochemical equivalents of the elements underlying the radioactive isotopes to be removed should not be selected. For example, K can be regarded as a biochemical equivalent of Cs. So, when it is intended to remove ¹³⁷Cs from the liquid, no K should be added. Likewise, Ca and Sr are to be understood to be biochemical equivalents.

Usefully, it is contemplated that one or more liquid samples are taken from the reactor and tested for their radioactivity and that, when a maximum value of the radioactivity is fallen below, the step of removing cleaned radioactive liquid from the reactor is performed.

In addition or alternatively, the method is further developed so that the step of removing cleaned radioactive liquid from the reactor is performed after the expiration of a predetermined period of time.

According to a further development of the method according to the invention, the approach is that, after the step of removing cleaned radioactive liquid from the reactor, the cleaned radioactive liquid is filtered and treated as a non-radioactive liquid.

Furthermore, the method is further developed so that, after the step of removing cleaned radioactive liquid from the reactor, the biomass is removed from the reactor and dehydrated as well as subsequently incinerated under heat supply.

According to a particularly advantageous embodiment, the approach is that the liquid obtained by the dehydration is tested for its radioactivity, wherein, when a maximum value of the radioactivity is fallen below, the liquid is treated as a non-radioactive liquid, and wherein, when a maximum value of the radioactivity is exceeded, the liquid is introduced into one or more earlier process steps in which the radioactive liquid present there is not or not yet sufficiently cleaned. In this way, a sufficient cleaning of the liquid can be achieved within a single procedural setup even though this cannot be guaranteed within a single process step.

It is advantageous that the working medium is stirred.

Particularly in case of purely aerobic microorganisms utilised in connection with the production of the preparation, it is contemplated that air is introduced into the working medium.

Similar to the cultivation of the aerobic microorganisms, the approach with regard to the supplied air is that the rate of introduced air in relation to one litre of the volume of the working medium is from 1 l/min to 2 l/min.

Usefully, the method is further developed so that a temperature of the working medium is kept in the range of a target temperature, particularly by temperature control.

It is particularly advantageous that the target temperature is in a range from 25 to 60° C. In case of the use of aerobic microorganisms, it has proven advantageous that the temperature is about 23 to 25° C. Otherwise, in case of anaerobic microorganisms, the temperature may be higher, for example about 55° C. When aerobic and anaerobic microorganisms are mixed a selection or variation between these thresholds is made.

Furthermore, it is advantageous that glucose and/or saccharose and/or ethanol and/or methanol are added as the substrate.

In this connection it is advantageous that the substrate is added in a concentration of 0.5 g/l/d to 2 g/l/d in relation to the volume of the working medium.

Furthermore, the invention relates to a method for cleaning a radioactively charged surface in which a preparation produced as described above is applied to the surface. Particularly radioactively charged environments of nuclear power plants are the target of this variant of the invention. The preparation is applied to the surfaces in dry form, watering the surfaces as required being an option to thereby promote a distribution on the surfaces or an entry of the preparation into them. The preparation may also be applied in a dissolved form, particularly in a water solution.

Therefore, the biological processing of the liquid radioactive waste is future-compliant both from an ecologic and from an economic point of view. The present invention relates to the processing of the liquid radioactive waste of lower and medium activity including a broad spectrum of radionuclides. Depending on the type and the properties of the waste, a biological structure of variable composition is used, namely adapted microorganism associations composed of bacteria and/or fungi and/or protozoa. The species composition of the microorganisms in the preparation varies and may contain up to 3000 or more strains. The effect of the preparation is based on the fact that life and growth of the microorganisms are possible in water solutions containing various salts, among them heavy metal and radionuclide salts, and having different pH values (from 4.5 to 9.0).

As is generally known, various chemical compounds dissolved in the water which are referred to as macro- and microelements here are required for the growth and reproduction of the microorganisms. The macroelements include: C, H, O, N, S, K, Ca, P, Mg, Fe. The microelements such as Mn, Mo, Zn, Cu, Co, Ni, Cl, Br, etc. are needed by the microorganisms in trace amounts. The biological processing of the liquid radioactive waste takes place during the growth of the microorganisms of the preparation in water solutions of the liquid radioactive waste. In the process, the microorganisms are activated by a rational change in the element composition in the liquid radioactive waste, the change in the ambient temperature, the breathing conditions (aerobic-anaerobic conditions), the introduction of specific substrates and other macro- and microelements. The rational change of the element composition in the medium means an exclusion of elements urgently required for the microorganism activity. Here, the radionuclides in the liquid radioactive waste have to function as biochemical equivalents of the excluded elements. In case of the biological processing of the liquid radioactive waste, elements having similar ion radii are to be regarded as biochemical equivalents.

### BRIEF DESCRIPTION OF THE DRAWINGS

The invention will now be explained by way of example with reference to the accompanying drawings with the aid of particularly preferred embodiments.

FIG. 1 shows a diagram for illustrating a method for the aerobic cultivation of microorganisms;

FIG. 2 shows a diagram for illustrating a method for the anaerobic cultivation of microorganisms;

FIG. 3 shows a diagram for illustrating of a method for cleaning radioactive liquids;

FIG. 4 shows a flow diagram for explaining a method for cleaning radioactive liquids.

### DETAILED DESCRIPTION OF THE INVENTION

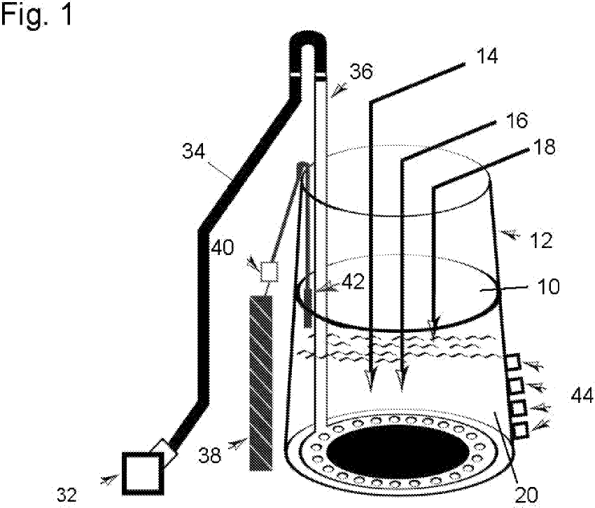

FIG. 1 shows a diagram for illustrating a method for the aerobic cultivation of microorganisms. For the aerobic cultivation von microorganisms, an aqueous solution 10 preferably having a pH value of 5.5 to 9.0 is provided in a container 12. The aqueous solution 10 is based on deionised water. To the aqueous solution 10, a substrate 14, particularly glucose is added. In addition, further elements are added, namely microelements and/or macroelements, the microelements being selected from the group of Mn, Mo, Zn, Cu, Co, Ni, Cl, Br, and the macroelements being selected from the group of C, H, O, N, S, K, Ca, P, Mg, Fe. Likewise, an inoculant 18 forming the basis of the biomass 20 to be cultivated is added. Outside of the container 12, a compressor 32 is provided which can introduce air into a jet nebuliser 36 through a pipe 34. The air is transported to the bottom of the container 12 so that it can be directly applied to the biomass 20 mainly present on the bottom of the container 12 there. Furthermore, a heating device 38 is disposed outside of the container 12. The heating device 38 is connected to a thermocouple 42 via a temperature controller 40 so that the temperature of the substances present in the container 12 can be controlled and/or regulated. Likewise, biomass collectors 44 are provided by means of which biomass 20 can be collected from the container 12.

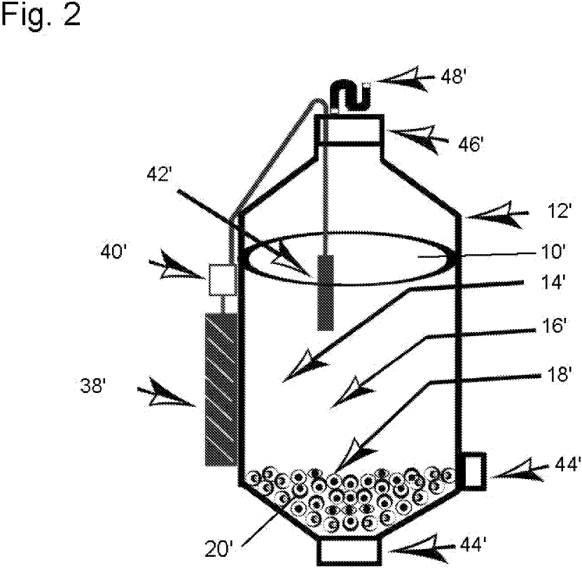

FIG. 2 shows a diagram for illustrating a method for the anaerobic cultivation of microorganisms. In the container 12′ for the anaerobic cultivation of microorganisms, again, an aqueous solution 10′ is contained, however, preferably having a pH value of 4.5 to 7.5. To the aqueous solution 10′, again, a substrate 14′, preferably glucose, other elements 16′, namely microelements and macroelements as indicated above, and an inoculant 18′ are added. Here as well, the inoculant 18′ is the initial substance for the biomass 20′ cultivated therefrom. Also, again a heating device 38′ is provided which is connected to a thermocouple 42′ via a temperature controller 40′, the heating device 38′ and the temperature controller 40′ being disposed outside of the container 12′ while the thermocouple 42′ is disposed in the container 12′ and particularly in the substances present in the container 12′ so that again the temperature of the substances can be controlled and/or regulated. The container 12′ further contains one or more biomass collectors 44′ for collecting biomass 20′. The container 12′ is hermetically closable by means of a closure 46′, particularly to prevent air from entering. In order to render the discharge of gases released within the container 12′ possible, a siphon-like water trap 48′ is provided on the upper side of the container 12′.

The preparation which is ultimately to be used for cleaning radioactive liquids and which is obtained on the basis of the described cultivation of microorganisms contains various groups of aerobic and anaerobic microorganisms, among them extremophiles which can exist under extreme conditions, namely at low and high temperatures (psychrophilies and thermophilies), in a high salt content (hallophilies) as well as at high radiation levels (radioresistant microorganisms).

The preparation of the preparation takes place by the non-sterile cultivation of various microbial primal associations originating from natural and technogenic sources by varying the growth conditions with the aim of obtaining associations characterised by a mutually beneficial coexistence. In addition, the climatic conditions of the intended place of use as well as the background composition of the primal microorganisms are taken into account in the preparation of the preparation. During the cultivation, apart from biogenic compounds, also such compounds which are toxic for microorganisms - in doses from harmless to semi-lethal - are contained in the solutions for the cultivation of the preparation. In addition, the solutions may contain the following substances: salts of heavy metals, petroleum products (among them polyaromatic hydrocarbons), surfactants, carbohydrates, protein, fats, etc. In an aggressive environment, non-surviving microorganisms of the preparation are a source for additional protection and nutrition for the surviving community.

A cultivation method according to the invention as the basis for the production of the preparation for cleaning radioactive waste can also be described as follows.

A first process step is the extraction of liquid (without biomass) on the occasion of the first addition of 1 ml substrate as well as the subsequent centrifugation at 14500 rpm for 3 minutes at ambient temperature. A content analysis is performed on the organic substance.

A second process step is the extraction of liquid (without biomass) 24 hours after the addition of 1 ml substrate including the subsequent centrifugation at 14500 rpm for 3 minutes at ambient temperature. A content analysis is performed on the organic substance.

Provided that, in the third process step, it is found that 90% of the organic substance was consumed, the next substrate dosage should be added within the course of 14 days (aerobic) or 50 days (anaerobic), regular content analyses of the organic substance and of the biogenic elements having to be carried out. In case there was no consumption of 90 percent the second process step is repeated.

Otherwise, the collection of 400 ml of biomass concentrate from a bioreactor as well as a centrifugation of the concentrate at 4000 rpm at 3 to 8° C. will be effected in the fourth process step (production stage). The excess of the centrifugation is returned into the bioreactor. The mass of moist biomass is determined, likewise the moisture content of the biomass.

In a fifth process step, the adapted biomass is ready for use if the mass of the centrifuged wet biomass is more than 50 grams per litre. Otherwise, the third process step and the following are repeated.

When the microbial association which, in particular, can be obtained by the described cultivation methods is thus ready for use compounds impeding the intended process are extracted from the biomass. The biomass is separated from the solution by centrifugation. If required, the biomass cultivated in various modes is mixed and washed three times with the isotonic solution on the basis of deionised water such as, for example, 0.1 to 0.9% NaCl solution in the ratio of 1:10 to 1:30. Then, it is centrifuged. The essence of this method is that the washing of biomass extracts compounds impeding the intended process of processing liquid radioactive waste.

A washing method within the scope of the production of the preparation for cleaning radioactive waste can also be described as follows.

From the containers in which the biomass is cultivated in the aerobic and anaerobic mode, the culture liquids are extracted and mixed in the ratio of 1:5 to 5:1 as well as centrifuged at 4000 revolutions per minute at a temperature of 3 to 8° C. for 8 to 12 minutes. The excess water is analysed for its elementary composition and introduced into a container for the recultivation of biomass.

The obtained wet biomass is added into isotonic solution in the ratio of biomass to solution of 1:4 bis 1:10 and thoroughly agitated for 5 to 10 minutes to avoid or eliminate a formation of clumps of the biomass.

The obtained solution is centrifuged at 4000 rpm at 3 to8° C. for 8 to 12 minutes. The excess water is discharged into the sewage system. The process is repeated, for example, three times.

In order to verify that the undesired elements were completely removed from the biomass, mass spectrometric analyses for the presence of elements are carried out on the initial excess and the third washing solution.

The washed moist biomass constituting the basis of the preparation for cleaning radioactive liquids is used for the processing of liquid radioactive waste either in a free form or in a special container or cartridge.

Special cartridges are hermetically closed containers made of a water-permeable non-woven fabric which let no microorganisms escape to the outside.

There is also the option to have the preparation prepared in the form of a dry powder, namely by lyophilisation. The approach for obtaining a dry powder including about 90% viable microorganisms is as follows.

Into the moist, washed, (after centrifuging) paste-like biomass, a cryoprotector is introduced, e.g. a water solution of dextran polysaccharide - 40% polyglucan solution in the ratio of 2.5% polyglucin in the finished paste and 2.5% glycerin (for 70 I of paste - 4.37 I of the 40 percent polyglucan solution and 1.75 I of glycerin). It is mixed for 10 minutes.

The finished mixture is frozen at -70° C.

The frozen mixture is subjected to lyophilic dehydration until a powder having a residual moisture of not more than 1 - 3% is formed.

This powder is tightly sealed in polyethylene bags under vacuum.

The properties of the preparation in the dry form remain unchanged for 3 years.

Therefore, the finished preparation consists of several thousands of microorganisms of various species adapted to life in hash conditions. Part of the microorganisms is capable of developing in aerobic and anaerobic conditions at a temperature of 20 to 80° C.

The cultivation and further processing of the microorganisms for the preparation does not require sterile conditions, expensive devices and reagents which renders keeping the original costs of the production of the preparation low possible.

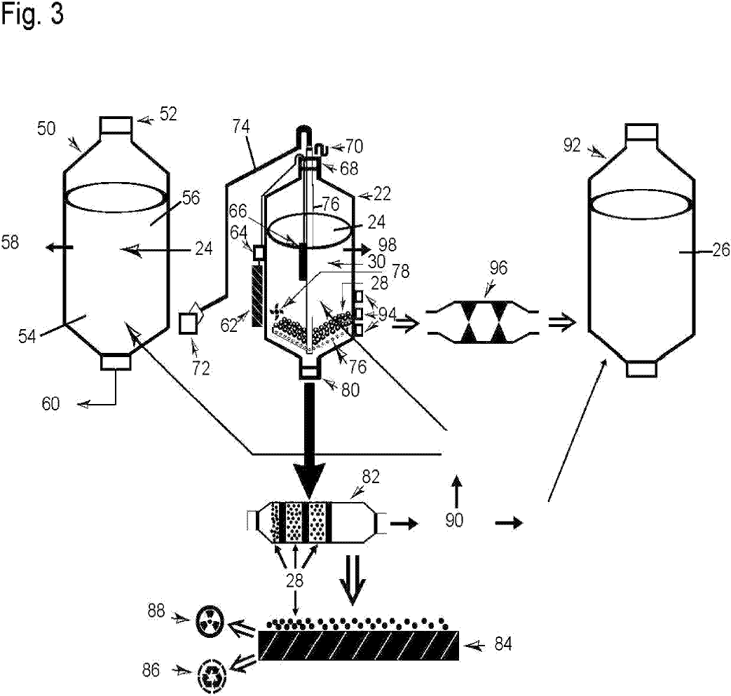

FIG. 3 shows a diagram for illustrating a method for cleaning radioactive liquids. A primary container 50 is provided in which radioactive liquid 24 to be cleaned is prepared. The radioactive liquid 24 to be cleaned is introduced into the container 50 through an opening 52 provided on the upper side of the primary container 50, along with, if required, deionised water 54 and macro- and microelements 56 as listed in detail above. The mixture or solution present in the primary container 50 is supplied to an analysis unit 58, it being decided based on the results of the analysis whether the mixture or solution is to be changed in its composition, for example to be diluted. As soon as the content of the primary container 50 has the desired properties, the content is completely or partly removed and supplied to a conveyor system 60. This conveyor system 60 conveys the content, i.e. the radioactive liquid 24 to be cleaned, into the reactor 22. Apart from the radioactive liquid 24 to be cleaned, a preparation 28 which was, particularly, obtained on the basis of the cultivation described in connection with FIGS. 1 and 2, as well as again a substrate 30, particularly glucose, are supplied to the reactor 22. Outside of the reactor 22, a heating device 62 is provided which is connected to a thermocouple 66 placed in the reactor 22 via a temperature controller 64 also disposed outside of the reactor 22. Thus, the temperature in the reactor 22 or of the substances present in the reactor 22 can be controlled and/or regulated. The reactor 22 is hermetically closable by a closure 68. In order to allow for gases released within the reactor 22 to be discharged, a siphon-like water trap 70 is provided on the upper side of the reactor 22. For the purpose of the air supply, a compressor 72 connected to a jet nebuliser 76 within the container via a pipe 74 is disposed outside of the reactor 22. Furthermore, an agitator 78 by means of which the substances in the reactor 22 can be stirred is disposed in the reactor 22. The content of the reactor 22 can be supplied to an analysing unit 98. Essentially, the reactor 22 is capable of processing aerobic and anaerobic microorganisms to clean the added radioactive liquid 24 based on them in this way. In case of a strictly anaerobic biomass, however, the means for supplying air, i.e. the compressor 72, the pipe 74, and the jet nebuliser 76, can, in principle, be omitted. The reactor 22 further comprises a biomass discharge system 80 through which the biomass 28 is suppliable to a filter press 82. In this filter press 82, the biomass 28 can be mechanically dehydrated, whereupon it is suppliable to a heating plate 84 to be incinerated there. Depending on whether the ash is below a radioactivity threshold, it can be supplied to a disposal 86 as low risk waste, or it is disposed of in a final disposal site 88 for the final storage of radioactive waste. The liquid emerging from the filter press 22 is supplied to an analysing unit 90. In this analysing unit, it will be determined whether the liquid is below a radioactivity threshold. If this is the case the liquid can be supplied to a waste water container 92 in which cleaned radioactive liquid 26, i.e. particularly no longer radioactive liquid or modestly radioactive liquid, from the filter press 82 is collected. However, if the radioactivity of the liquid from the filter press 82 exceeds a predetermined radioactivity threshold, it is returned to one of the preceding process stages, i.e., in particular, supplied to the primary container 50 or the reactor 22. The waste water container 92 is also supplied with the liquid freed from radioactivity from the reactor 22, namely through the reactor’s on-site discharge system 94 for cleaned liquid as well as a coarse cleaning filter 96.

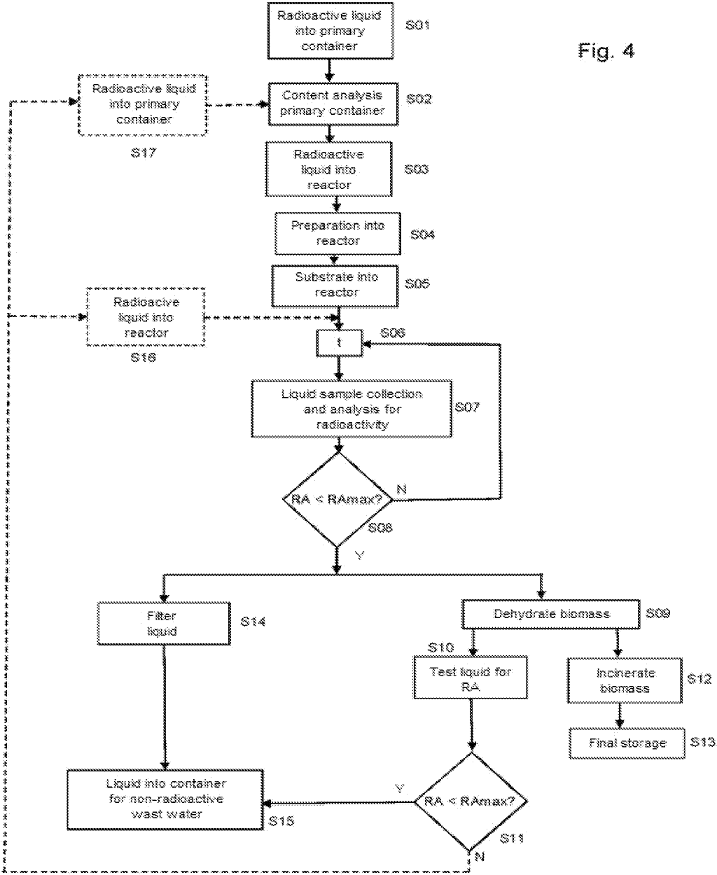

FIG. 4 shows a flow diagram for explaining a method for cleaning radioactive liquids. In step S01, radioactive liquid is made available in a primary container. The content of the primary container is analysed in step S02, a change of the content depending on the results of the analysis still being possible, for example by the targeted addition of macroelements and/or microelements and/or of additional deionised water for dilution. In step S03, the radioactive liquid is filled into the reactor. In step S04, the preparation for cleaning is supplied, and in step S05 the substrate, i.e. particularly glucose. Subsequently, according to step S06, there is a waiting time of a period of time t. In step S07, a liquid sample is taken and analysed for radioactivity. If, in step S08, it is determined that the radioactivity (RA) has not yet fallen below a maximum radioactivity (RAmax), the course of the process will be returned to step S06, and there is another specified waiting time. However, when the radioactivity has fallen below a maximum radioactivity (RAmax), the liquid can be removed from the reactor. It is filtered in step S14, and filled into a container for non-radioactive waste water in step S15. The biomass is also removed from the reactor and dehydrated in step S09. The dehydrated biomass is incinerated in step S12 and permanently stored in step S13. Instead of a final storage, it can also be disposed of as low risk waste in case of a sufficiently low or no longer detectable radioactivity of the incinerated biomass. The liquid resulting from the dehydration performed in step S09 is analysed for its radioactivity in step S10. In step S11, it is tested whether the radioactivity (RA) is smaller than a maximum radioactivity (RAmax). If this is the case the liquid can be filled into a container for non-radioactive waste water according to step S15. If the radioactivity (RA) is not lower than a maximum radioactivity (RAmax) the liquid is returned to the primary container according to step S17 or to the reactor according to step S16.

For cleaning purposes, the radioactive waste is thus introduced into a special reactor enabling a thermostat function for its content as well as ventilation and/or stirring with air and/or an agitator. In the reactor, there is also the preparation, a set of macro- and microelements and a substrate for the growth of the culture. The process takes place at a temperature of 20 to 80° C. In case of aerobic cultures, flowing air from an external compressor is supplied through a tube having a weight and a nebuliser at the outlet at a rate of 1.5 bis 3.0 I/min per one litre of the liquid radioactive waste for ventilation. The duration of the ventilation and/or stirring varies from continuously to periodically depending on the processing objective. For example, the duration may be 20 s per day. In the process, the preparation should be stirrable from the bottom to the surface; however, a leakage of the biomass on the upper container walls (not covered by liquid) in the course of the method is to be avoided. The consumption of the preparation varies from 1 bis 35 g moist biomass (moisture 93 - 99%) or 0.01 - 2.45 g lyophilised powder per one litre of the liquid radioactive waste. As the substrate, for example, highly pure glucose is used which is added in an amount of 0.5 to 5 g per one litre of the liquid radioactive waste per day. As the biogenic elements, K, Ca, P, S, N, Zn, Co, Mn, Cl, Cu, Mo, Ni, Se, B, Fe are introduced. However, to achieve the conditions of an accelerated biosorption, particularly in case of anaerobic cultures, or to reduce the activity the solution, particularly in case of aerobic cultures, one or some elements should be missing in the medium to be processed if they are stoichiometric equivalents of the radionuclide to be processed which is used for growth by the microorganism cells.

Depending on the processing objectives, the liquid radioactive waste is converted into solid radioactive waste with a multiple reduction of volume, or the activity of the liquid radioactive waste is directly reduced.

The liquid radioactive waste is first supplied to a preparation container or primary container. Samples are taken to carry out necessary analyses, e.g. with the following methods:
mass spectrometry (identification of stable and radioactive elements);
ion chromatography by means of a conductometric detector;
potentiometry.

The effect of the preparation was, in particular, tested under the following conditions:
radioactivity of the solution lower than 2·10² kBq/l;
overall salt content lower than 100 g/l;
pH value of the medium from 5 to 9.

After having performed the examinations, it is therefore recommended to dilute the liquid radioactive waste with deionised water when required. Then, macro- and microelements are introduced into the primary container when required.

It is important to emphasise that the liquid radioactive waste should not contain any stable isotopes of the radionuclides to be disposed of so as to be capable of successfully reducing the radioactivity. For growth, the microorganisms will, first of all, absorb the stable isotopes of the radionuclides to be disposed of. From the mixture ¹³³Cs and ¹³⁷Cs, e.g., the preparation will first process the stable isotope ¹³³Cs. Consequently, the radioactivity of the liquid radioactive waste will not change in the process.

The prepared liquid radioactive waste is supplied into the reactor via the conveyor system.

Then, the preparation and the substrate for the growth of the microbiological cultures are added into the reactor. While the ventilation and/or stirring are deactivated, the reactor lid is tightly closed. The lid is equipped with a liquid seal filled with deionised water and discharging excess gas from the reactor when required. The temperature in the reactor is maintained at the target level by means of a heating element and a temperature control encoder. The temperature of the medium in the reactor is adjusted with the aid of a thermocouple. The ventilation takes place with oxygen from the ambient air which is supplied via the compressor and the jet nebuliser. The stirring is carried out by a built-in propeller. Regularly, a sample is taken from the upper liquid layer to determine the radioactivity. When it is the objective to rapidly store the radionuclides in the microorganisms for further final storage the maximum biosorption is reached within 1 to 7 days. If it is the objective to reduce the radioactivity of the solution while no final storage of the radioactive substances is intended it is required to maintain the fast growth of the microorganisms until the radioactivity is reduced to the background or target level.

When the objective of cleaning the water solution from the radionuclides is reached the liquid cleared of the radionuclides flows into the containers for the non-radioactive waste water via the discharge system and a coarse filter, e.g. a sand filter, and the spent biomass of the preparation is transferred onto the filter press by the biomass-discharge system. If a cartridge is used the cartridge including the spent biomass is removed from the water, then mechanically pressed out above the water surface according to the tea bag principle, dried in air, and then opened to place the content (spent biomass) on the heating plate and to bypass the filter press.

If the preparation is used in a loose form (without a cartridge) the spent biomass having a moisture of about 93 - 99% is mechanically dehydrated in the filter press to the largest possible extent and then placed on the heating plate for a slight incineration in air at a temperature of not more than 250° C. In the process, the liquid separated from the biomass in the filter press is examined for radioactivity. If the radioactivity of the filtrate does not exceed the target values the liquid is also discharged into the containers for the non-radioactive waste water. Otherwise, the liquid flows back into the reactor or into the primary container for the preparation of the liquid radioactive waste to be processed with the next batch of the liquid radioactive waste. If a reduction of the radioactivity below specific target values is not required or if no further batch of liquid radioactive waste is in the queue for cleaning the radioactive filtrate the volume of which does not exceed the volume of the spent moist biomass may be evaporated onto the heating plate for the further final storage of the residual salts.

When the biosorption of the radionuclides is applied the generated ash including the radionuclides stored in it is forwarded for final storage. In case of a reduction of the radioactivity, the ash poses no risk to humans, plants, and animals and can be disposed of according to traditional methods.

When the objective of clearing the water solution of the radionuclides by sorption was not reached in the first application of the preparation it is required to replace the spent preparation by new preparation. Here, the liquid radioactive waste and the spent biomass are processed as described above. The method of biosorption should be repeated until the radioactivity of the solution is reduced to the background or target level.

In the final stage of the processing of the liquid radioactive waste by means of the method of biosorption by means of the preparation, low risk “technical” water and completely dry ash of the microorganisms including the radioactive elements stored in it are generated. In this way, a multiple reduction of the amount of waste for final storage is achieved. In case of a successful transformation of the radionuclides into stable elements, no final storage of the waste is required.

The association of microbes existing on the edge of survival incorporates radionuclides into the cells. In the process, the microorganisms grow by lysis of some cells and discharge of remaining amounts of deficiency macroelements into the water medium. In the biological systems, the atomic transformations take place in nanoscale spaces of the microorganism cells. For the protons, the nanoscale spaces of the growing biological cells represent potential wells having dynamically changing walls creating coherent correlated states of the quantum particles. The protons existing in these states are capable of a reaction resulting in the formation of elements required for the further performance of biochemical processes in the microorganisms. Therefore, the active division of the cells including the radionuclides in the nanoscale spaces results in the reduction of the radioactivity under formation of the stable elements from the radionuclides.

In the present context, the phenomenon of the transmutation of atomic nuclei by nucleosynthesis may play a role. For nucleosynthesis, a number of requirements has to be fulfilled. One of them is associated with the necessity of incorporating the synthesising isotope in the metabolism as fast as possible including the associated fixation as a stable nucleus (stable atom). It is obvious that the biochemical processes supporting storage simultaneously promote the transmutation of atomic nuclei. During the growth of microbiological cultures, the reproduction of DNA molecules and some other macromolecules, their orientation, and the formation of the primary and secondary structures take place. In the area of the structural adaptation, a continuous process of the formation and modification of microinhomogenities with a characteristic magnitude of the same order takes place which is required for the “elimination” of the Coulomb barrier in the transmutation of atomic nuclei. If the nutrient medium for the microbiological culture does not contain an isotope of the element required for the growth of the culture, but the other isotopes which may constitute the isotope required for the growth as result of the transmutation of atomic nuclei this isotope is, upon formation as the result of a reaction, immediately absorbed by and fixed in the microbiological culture in optimum micro activities. Therefore, there is the possibility to utilise the transformation of the radioactive isotopes absorbed by the bacteria culture into stable isotopes of other chemical elements. In this case, this does not refer to a comprehensive use of microbiological systems in the first stages of the processing of spent fuel elements and highly active waste.

In the following, some examples for illustrating the invention are provided:

Example 1: As a model for waste water including heavy metals, deionised water in a volume of 75 ml with K₂Cr₂O₇ dissolved therein in a concentration of 0.1 g/l and with an overall salt content of about 100 g/l was used. Introduced into the reactor were: A set of macro- and microelements with the exception of biochemical chromium equivalents, then moist paste-like preparation in an amount of 2.21 g as well as a substrate in the ratio of 5 to 6 g organic substance per one litre of the mixture. The experiment was predominantly carried out under microaerophilic conditions at a temperature of 55 to 80° C. In the course of the experiment, the pH value of the medium changed in the range of 6.9 to 7.9. The consumption level of the organic substance and the relevant macro- and microelements were monitored. Within a maximum of 7 days, the initially coloured solution became completely colourless. Spectrophotometric examinations still revealed traces of chromium in the solution.

Example 2: Here, the approach was like in Example 1 with the exception that the compound K₂Cr₂O₇ was used in a concentration of 10 g/l. In this case, the solution became colourless within 50 days of the experiment. Spectrophotometric examinations again revealed traces of chromium in the solution. The preparation had a bluish tint.

Example 3: As a model for liquid radioactive waste, deionised water in a volume of 75 ml with radioactive caesium having an activity of 180 kBq/l dissolved in the water was used. Introduced into the reactor were: A set of macro- and microelements with the exception of biochemical caesium-equivalents, then moist paste-like preparation in an amount of 2.5 g as well as a substrate in the ratio of 3 to 4 g organic substance per one litre of the mixture. The experiment was predominantly carried out under microaerophilic conditions at a temperature of 55 to 65° C. In the course of the experiment, the pH value of the medium changed in the range from 5.4 to 7.5. Within 20 days, the activity of the liquid was reduced down to 20 kBq/l.

Example 4: As a model for liquid radioactive waste, deionised water in a volume of 750 ml with the compound CsNO₃ in a concentration of 0.5 g/l dissolved in the water was used. Introduced into the reactor were: A set of macro- and microelements with the exception of biochemical caesium-equivalents, then moist paste-like preparation in an amount of 9.55 g as well as a substrate in the ratio of 0.5 bis 1 g organic substance per one litre of the mixture. The experiment was predominantly carried out under aerobic conditions at a temperature of 20 to 28° C. In the course of the experiment, the pH value of the medium changed in the range from 6.1 to 7.9. The consumption level of the organic substance and the relevant macro- and microelements were monitored. Within a maximum of 7 days, mass spectrometric analyses revealed a transition from Cs into the biomass. The caesium content in the biomass was up to 300 mg/g of the completely dry biomass.

Example 5: As a model for liquid radioactive waste, deionised water in a volume of 75 ml with the radioactive caesium-compound CsNO₃ dissolved in the water was used which guarantees a caesium content at a level of 5·10⁴ Bq. Introduced into the reactor were: A set of macro- and microelements with the exception of biochemical caesium equivalents, then moist paste-like preparation in an amount of 0.9 g. No substrate was introduced into the bioreactors. The experiment was predominantly carried out under aerobic conditions at a temperature of 20 to 25° C. In the course of the experiment, the pH value of the medium changed in the range from 6.1 to 7.2. The biomass was removed from the reactor every 1 to 3 days. Radiospectroscopic examinations of the biomass revealed a caesium content in the moist biomass of up to 17 kBq per one gram of the completely dry biomass.

Example 6: As a model for liquid radioactive waste, deionised water in a volume of 700 ml with a compound of stable caesium CsNO₃ in a concentration of 0.12 g/l dissolved in the water was used. The preparation was introduced into the reactor so that the bioreactor contained 10 g of the moist biomass and a set of macro- and microelements with the exception of biochemical caesium equivalents as well as a substrate in the ratio of 2 to 3 g organic substance per one litre of the mixture. The experiment was predominantly carried out under microaerophilic conditions at a temperature of 35 to 45° C. The best result was already achieved after 8 days of the experiment. An analysis of the content of the bioreactor by means of atomic emission spectrometry with an inductively coupled plasma revealed that the caesium content was reduced by 55%. In the bioreactors, barium was detected which may indicate a transmutation of atomic nuclei.

Example 7: As a model for liquid radioactive waste, deionised water in a volume of 750 ml with the radioactive caesium compound CsNO₃ dissolved in the water was used which guarantees a caesium content on a level of 10⁴ Bq. Introduced into the reactor were: A set of macro- and microelements with the exception of biochemical caesium equivalents, then moist paste-like preparation in an amount of 9.05 g as well as a substrate in the ratio of 0.5 bis 1 g organic substance per one litre of the mixture. The experiment was carried out under aerobic conditions at a temperature of 20 to 28° C. In the course of the experiment the pH value of the medium changed in the range from 6.5 to 8.1. The consumption level of the organic substance and the relevant macro- and microelements were monitored. Within a maximum of 15 days, radiospectroscopic examinations revealed an average reduction of the activity of the content of the bioreactor by 23%.

If radionuclides from water solutions are to be rapidly concentrated the preparation seems to be a universal biosorbent which is easy to dispose of and which is capable of extracting all substances in arbitrary quantity ratios from the liquid radioactive waste. In case of a rational change of the element composition in the medium and during the growth of the microorganisms, the preparation is capable of transforming predetermined elements and thus transform the radioactive elements from the liquid radioactive waste into non-radioactive elements.

In the biological processing of liquid radioactive waste under application of the preparation, no extreme temperatures or pressures are required so that the method requires a low energy intensity. Furthermore, the application of the preparation does not require the introduction of hazardous chemical reagents which excludes the risk of a secondary contamination. The features described above guarantee utmost simplicity and operational safety of the procedural facility.

The features of the invention disclosed in the above description, in the drawings as well as in the claims may be important for the realisation of the invention both individually and in any combination.

List of numerals

| | |
|---|---|
| 10 | Aqueous solution |
| 10′ | Aqueous solution |
| 12 | Container |
| 12′ | Container |
| 14 | Substrate |
| 14′ | Substrate |
| 16 | Other elements |
| 16′ | Other elements |
| 18 | Inoculant |
| 18′ | Inoculant |
| 20 | Biomass |
| 20′ | Biomass |
| 22 | Reactor |
| 24 | Radioactive liquid |
| 26 | Radioactive liquid |
| 28 | Biomass |
| 30 | Substrate |
| 32 | Compressor |
| 34 | Pipe |
| 36 | Jet nebuliser |
| 38 | Heating device |
| 38′ | Heating device |
| 40 | Temperature controller |
| 40′ | Temperature controller |
| 42 | Thermocouple |
| 42′ | Thermocouple |
| 44 | Biomass collector |
| 44′ | Biomass collector |
| 46′ | Closure |
| 48′ | Water trap |
| 50 | Primary container |
| 52 | Opening |
| 58 | Analysis unit |
| 60 | Conveyor system |
| 62 | Heating device |
| 64 | Temperature controller |
| 66 | Thermocouple |
| 68 | Closure |
| 70 | Water trap |
| 72 | Compressor |
| 74 | Pipe |
| 76 | Jet nebuliser |
| 78 | Agitator |
| 80 | Biomass discharge system |
| 82 | Filter press |
| 84 | Heating plate |
| 86 | Disposal |
| 88 | Final disposal site |
| 90 | Analysing unit |
| 92 | Waste water container |
| 94 | Discharge system |
| 96 | Coarse cleaning filter |
| 98 | Analysing unit |

1. A method for the aerobic cultivation of microorganisms, comprising the steps of:
providing an aqueous solution having a pH value of 5.5 to 9.0 in a container,
adding a substrate in a first substrate dosage to the aqueous solution,
adding further elements to the aqueous solution,
adding an inoculant with microorganisms to the aqueous solution,
wherein an initial product is provided by the abovementioned steps, and wherein, subsequently, the following steps are performed:
introducing air into the initial product or into intermediate products developing therefrom,
varying a temperature of the initial product or the intermediate products developing therefrom in a range from 15 to 35° C.,
wherein, after having added the substrate in the first substrate dosage, the following steps are performed for monitoring the cultivation:
taking a reference liquid sample and determining a first concentration of organic substance in the reference liquid sample,
taking another liquid sample and determining another concentration of organic substance in the further liquid sample after the expiration of the first waiting time,
if the further concentration of organic substance is smaller than 10 percent of the first concentration of organic substance, adding substrate in another substrate dosage,
repeating the abovementioned steps for monitoring the cultivation until a sufficient amount of biomass is present in the container.

2. (canceled)

3. The method according to claim 1, wherein, a sufficient amount of biomass is present in the container when 50 g of wet biomass are extractable from one litre of product volume, wet biomass having a moisture content of 93 to 99 percent.

4. The method according to claim 1, wherein the aqueous solution is based on deionised water.

5. The method according to claim 1, wherein glucose or saccharose or ethanol or methanol are used as the substrate.

6. The method according to claim 1, wherein the first substrate dosage is added in a concentration of 0.5 g/l/d to 2 g/l/d, claim 1, and the further substrate dosages are added in a concentration of 0.5 g/l/d to 2 g/l/d, respectively in relation to the volume of the aqueous solution.

7. The method according to claim 1,wherein microelements or macroelements are added as further elements, wherein the wherein the microelements are selected from the group of Mn, Mo, Zn, Cu, Co, Ni, Cl, Br, and wherein the macroelements are selected from the group of C, H, O, N, S, K, Ca, P, Mg, Fe.

8. The method according to claim 6, wherein the microelements are added in a concentration of 0.5 ^{∗} 10-5 g/l to 2 ^{∗} 10-5 g/l and the macroelements in a concentration of 0.5 g/l to 2 g/l, respectively in relation to the volume of the aqueous solution.

9. The Method according to claim 1 wherein the inoculant contains bacteria claim 1, or fungy or protozoa.

10. The method according to claim 1, wherein the inoculant is added in a concentration of 5 to 10 percent in relation to the volume of the aqueous solution.

11. The method according to claim 1, wherein air is introduced during predetermined periods of time and that no air is introduced outside of these predetermined periods of time.

12. The method according to claim 11, wherein the rate of introduced air in relation to one litre of the volume of the initial product is from 1 l/min to 2 l/min.

13. A method for the production of a preparation for cleaning radioactive liquids and surfaces, comprising the steps of:
providing of a substance with aerobic microorganisms, cultivated using a method aerobic of cultivation of microorganisms comprising the steps of:
providing an aqueous solution having a pH value of 5.5 to 9.0 in a container.
adding a substrate in a first substrate dosage to the aqueous solution,
adding further elements to the aqueous solution,
adding an inoculant with microorganisms to the aqueous solution,
wherein an initial product is provided by the abovementioned steps, and wherein, subsequent, the following steps are performed:
introducing air into the initial product or into intermediate products developing therefrom,
varying a temperature of the ínitial product or the intermediate products developing therefrom in a range from 15 to 35 degree Celsius,
wherein, after having added the substrate in the first substrate dosage, the following steps are perfomed for monitoring the curtivation:
taking a reference liquid sample and determining a first concentration of organic substance in the reference liquid sample.
taking another liquid sample and determining another concentration of organic substance in the further liquid sample after the expiration of the first waiting time,
if the further concentration of organic substance is smaller than 10 percent of the first concentration of organicsubstance, adding substrate in another substrate dosage.
repeating the abovementioned steps for monitoring the cultivation until a sufficient amount of biomass is present in the container, or
providing of a substance with anaerobic microorganisms, particularly cultivated according to one of the claims 2 to 10 cultivated using a method for the anaerobic cultivation of microorganisms, comprising the steps of:
providing an aqueos solution having a pH value of 4.5 to 7.5 in a container
adding a substrate in a first substrate dosage to the aqueous solution,
adding further elements to the aqueous solution,
adding an inoculant with microorganisms to the aqueous solution,
wherein an initial product is provided by the abovementioned steps, and wherein, subsequently, the following steps are performed:
hermetically sealing the container,
varying a temperature of the initial product or the intermediate products developing therefrom in a a range from 40 to 80° C.,
wherein, after having added the substrate in the first substrate dosage, the following steps are performed for monitoring the cultivation:
taking a reference liquid sample and determining a first concentration of organic subtance in the reference liquid sample,
taking another liquid sample and another concentration of organic substance in the further liquid sample after the expiration of the first waiting time,
if the further concentration of organic substance is smaller than 10 percent of the first concentration of organic substance, adding substrate in another substrate dosage,
repeating the abovementioned steps for monitoring the cultivation until a sufficient amount of biomass is present in the container,
wherein the substance is subjected to the following steps for washing:
centrifuging the substance for producing wet biomass,
adding an isotonic solution to the wet biomass for producing a washed substance, wherein the steps for washing are repeated a plurality of times, if required, to thereby provide the preparation for cleaning radioactive liquids and surfaces.

14. The method according to claim 13, wherein the substance with aerobic microorganisms and the substance with anaerobic microorganisms are mixed prior to being washed.

15. The method according to claim 13, wherein, after washing, an analysis of the washed biomass is carried out.

16. The method according to claim 13, wherein the preparation for cleaning radioactive liquids and surfaces is filled into water-permeable cartridges in portions.

17. The method according to claim 13, wherein the preparation for cleaning radioactive liquids and surfaces is dried by means of lyophilisation.

18. A method for cleaning radioactive liquids, comprising the steps of:
providing radioactive liquid to be cleaned in a reactor,
adding a preparation for cleaning radioactive liquids into the reactor, the preparation produced by a method for the production of a perparation for cleaning radioactive liquids and surfaces comprising
providing of a substance with aerobic microorganisms, particularly cultivated comprising the steps of:
providing an aqueous solution having a pH value of 5.5 to 9.0 in a container
adding a substrate in a first substrate dosage to the aqueous solution,
adding further elements to the aqueous solution,
adding an inoculant with microorganisms to the aqueous solution,
wherein an initial product is provided by the abovementioned steps, and wherein, subsequently, the following steps are performed:
introducing air into the initial product or into intermediate products developing therefrom,
varying a termperature of the initial product or the intermediate products developing therefrom in a range from 15 to 35 degrees Celsuis,
wherein, after having added the substratae in the first substrate dosage, the following steps are performed for monitoring the cultivation:
taking a reference liquid sample and determining a first concentration of organic substance in the reference liquid sample, substance in the reference liquid sample,
taking another liquid sample and determining another concentration of organic substance in the further liquid sample after the expiration of the first waiting time,
if the further concentration of organic substance is smaller than 10 percent of the first concentration of organic susbtance, adding substrate in another substrate dosage,
repeating the abovementioned steps for monitoring the cultivation until a sufficient amount of biomass is present in the container, or
providing of a substance with anaerobic microorganisms, particularly cultivated the anaerobic cultivation of microorganisms comprising the steps of:
providing an aqueous solution having a pH value of 4.5 to 7.5 in a container,
adding a substrate in a first substrate dosage to the aqueous solution,
adding further elements to the aqueous solution,
adding an inoculant with microorganisms to the aqueous solution, wherein an initial product is provided by the abovementioned steps, and wherein, susbsequently, the following steps are performed:
hermetically sealing the container,
varying a temperature of the initial product or the intermediate products developing therefrom in a range from 40 to 80° C.,
wherein, after having added the substrate in the first substrate dosage, the following steps are performed for monitoring the cultivation:
taking a reference liquid sample and determining a first concentration of organic substance in the reference liquid sample,
taking another liquid sample and determining another concentration of organic substance in the further liquid sample after the expiration of the first waiting time,
if the further concentration of organic substance is smaller than 10 percent of the first concentration of organic substance, adding substrate in another substrate dosage,
repeating the abovementioned steps for monitoring the cultivation until a sufficient amount of biomass is present in the container.
wherein the substance is subjected to the following steps for washing:
centrifuging the substance for producing wet biomass.
adding an isotonic solution to the wet biomass for producing a washed substance, wherein the steps for washing are repeated a plurality of times, if required, to thereby provide the preparation for cleaning radioactive liquids and surfaces,
adding a substrate into the reactor,
adding elements selected from the group of K, Ca, P, S, N, Zn, Co, Mn, Cl, Cu, Mo, Ni, Se, B, Fe,
wherein, by the abovementioned steps, a working medium is provided, and wherein, further, the following step is performed:
removing cleaned radioactive liquid from the reactor.

19. The method according to claim 18, wherein a selection from the group of K, Ca, P, S, N, Zn, Co, Mn, Cl, Cu, Mo, Ni, Se, B, Fe of added elements is depleted of one or more elements which are to be understood to be biochemical equivalents of the elements on which the radioactivity of the radioactive liquid is based.

20. The method according to claim 18, wherein one or more liquid samples are taken from the reactor and tested for their radioactivity, and in that, when a maximum value of the radioactivity is fallen below, the step of removing cleaned radioactive liquid from the reactor is performed.

21. The method according to claim 18, wherein the step of removing cleaned radioactive liquid from the reactor is performed after the expiration of a predetermined time.

22-32. (canceled)
